(19) United States
(12) Patent Application Publication
(10) Pub. No.: US 2016/0068809 A1
(43) Pub. Date: Mar. 10, 2016

(54) USE OF ZEBURALINE FOR THE TREATMENT OF AUTOIMMUNE DISEASES OR IMMUNE REJECTION OF TRANSPLANTS

(71) Applicant: Idogen AB, Lund (SE)

(72) Inventors: Leif G. Salford, Lund (SE); Bertil Persson, Lund (SE); Hans Olov Sjögren, Lund (SE); Bengt Widegren, Lund (SE)

(21) Appl. No.: 14/940,657

(22) Filed: Nov. 13, 2015

Related U.S. Application Data
(63) Continuation of application No. 12/601,021, filed on Aug. 23, 2011.
(60) Provisional application No. 60/933,478, filed on Jun. 7, 2007.

(30) Foreign Application Priority Data
May 25, 2007 (SE) 0701275-0

(51) Int. Cl. C12N 5/0783; A61K 39/00

(52) U.S. Cl. CPC C12N 5/0636; A61K 39/001; A61K 39/0008; A61K 2039/577; A61K 2039/5154; C12N 2501/999; C12N 2501/22; C12N 2501/73

(57) ABSTRACT

The invention relates to the use of 1-(β-D-Ribofuranosyl)-1,2-dihydropyrimidin-2-one derivative or mimetic or an analogue, derivatives, metabolites, variants or salts thereof for the manufacturing of a medicament to increase the amount of Indoleamine 2,3-dioxygenase (IDO) production in order to induce immunological tolerance as well as a method of treating a mammal in need thereof.

### FIELD OF INVENTION

The invention relates to the use of zebularine(1-(β-D-Ribofuranosyl)-1,2-dihydropyrimidin-2-one), analogue, derivative or mimetic or salts thereof for the manufacturing of a medicament to increase the amount of Indoleamine 2,3-dioxygenase (IDO) production in order to induce immunological tolerance as well as a method of treating a mammal in need thereof.

### BACKGROUND OF INVENTION

Organ transplantation is currently the treatment of choice for end-stage kidney, heart and liver diseases and is increasingly performed also in patients with failing other organs. However, despite advances in the often immunosuppressive therapy and prophylaxis of infectious complications, rejection and severe infections still remain as problems after organ transplantation. In gene therapy, genes are in vivo transferred into viable cells of patients or in vitro whereafter the modified cells are transplanted to the patient resulting in continued production of the corresponding proteins as long as the producing cells survive and the transferred genes are not lost or silenced, e.g. by DNA methylation. As a rule additional proteins related to the vector used for gene transfer are also expressed.

These proteins are foreign to the patient whose immune system mounts an immune response resulting in a high risk of killing of the expressing cells and loss of effect of the gene therapy.

It is well recognized that the optimal way of avoiding these problems of rejection of cells or organs expressing foreign antigens is to induce immunological tolerance to these antigens. In cases where an antigen selective non-responsiveness is physiologically developing, it is often dependent on a functioning IDO and administration of inhibitors of IDO breaks the state of unresponsiveness (Miki et al., 2001). Transfer of the IDO gene into allogeneic cells can lead to over expression of IDO and has been reported to result in a strong suppression of the rejection response to the allografts and permanent survival of the transferred cells without supporting immunosuppressive therapy.

Autoimmune diseases are widely spread and are causing a substantial proportion of the chronical illnesses in man. There is a great number of different autoimmune diseases, the most common being Rheumatoid arthritis, Diabetes type I, Psoriasis, Sjögrens syndrome, Multiple Sclerosis (MS), Crohns disease. There also exists a number of degenerative diseases that are likely to have autoimmune components and among these can be mentioned, arteriosclerosis, Parkinson's disease, ALS (Amyotrophic lateral sclerosis), dementia. In addition, there are situations when the immune system reacts very strongly in trauma like stroke and heart infarction and where the insult caused by lack of oxygen is enhanced by toxicity from the secondary immune reactivity. Another such situation where the immune system causes damage is after transplantation or gene therapy. The rejection of the transplant is mediated by the immune system and transplanted or genetically treated patients need rather strong immune suppressive drugs for the rest of there lives and these immune suppressive drugs cause several severe side effects. Zebularine is an cytidine analogue, and a DNA methyl transferase inhibitor that has been shown to be stable and not very toxic compared to azacytidine 2′-deoxy-azacytidine (Cheng et al. 2003). Inhibitors of DNA metyl transferases have been used in preclinical and clinical trials as therapies against cancer. Genes inhibited by methylation that can favour tumour growth are tumour suppressor genes inducing genes, anti- an genes, immune-stimulatory genes, and tumour antigens. In human bladder cancer zebularine 100-500 μM) induces p16 gene expression (Cheng et al. 2004a) and when the bladder carcinoma cells grown in BALB/c mice were treated with zebularine (500-1000 mg/kg), tumour volume was reduced, and an in vivo p16 gene expression was induced (Cheng et al. 2004a), Zebularine can also change the expression of some other genes in cancer cells such as the tumour antigen MAGE-1 that is important to interact with the immune system (Cheng et al, 2004b, Liu et al. 2004.

Indoleamine 2,3-dioxygenase (IDO) degrades the indole moiety or tryptophan and initiates the production of neuroactive and immunoregulatory metabolites, collectively known as kynurenines. The functional expression of IDO by dendritic cells has emerged in recent years as a major mechanism of peripheral tolerance. IDO contributes to maternal tolerance in pregnancy, control of allograft rejection, and protection against autoimmunity, inflammatory pathology and allergy. IDO expression also serves a physiological mechanism by which malignancies induce immune tolerance (Uyttenhove et al, 2004; Mellor et al. 2004; Munn et al 2004;). The wide spectrum of physiopathological conditions in which IDO appears at work, suggests that this suppressive system is frequently involved in physiological down regulation of T cell responses and resulting inflammatory responses.

### SUMMARY OF THE INVENTION

The invention relates to the use of zebularine(1-(β-D-Ribofuranosy)-1,2-dihydropyrimidin-2-one), analogue, derivative or mimetic or salts thereof for the manufacturing of a medicament to increase the amount of Indolearnine 2,3-dioxygenase (IDO) production in order to induce immunological tolerance as well as a method of treating a mammal in need thereof. Indolamine dioxygenase(IDO) produces catabolites and metabolites from tryptophan see below. The medicament of the invention induces immunological tolerance in a mammal and may be used to treat a number of disorders and diseases, such as those mentioned below.

Additionally, the invention relates to the use of at least zebularine, derivative or mimetic or an analogue or salts thereof for the manufacturing of a medicament for the treatment of an autoimmune disorder or disease or immune rejection of transplants or gene therapeutically modified cells, wherein the treatment induces indolamine dioxygenase.

By providing such a medicament it is possible for the first time to induce immunological tolerance in an efficient way and thereby to enable the possibility to treat a number of diseases as defined below.

Zebularine, derivative or mimetic or an analogue, or salts thereof may be used for the manufacturing of a medicament for the treatment of a disease selected from the group consisting of Achlorhydria, Acute hemorrhagic leukencephalitis, Addison's Disease, Alopecia Areata, Anemia, Pernicious Anti-Glomerular Basement Membrane Disease, Antiphospholipid Syndrome, Aplastic Anemia, Atopic Allergy, Autoimmune Atrophic Gastritis, Autoimmune Hearing Loss, Autoimmune hemolytic anemia, Autoimmune hypoparathyroidism, Autoimmune hypophysitis, Autoimmune Lymphoproliferative, Autoimmune Myocarditis, Autoimmune oophoritis, Autoimmune orchitis, Autoimmune Polyendocrinopathy-Candidiasis-Ectodermal-Dystrophy, Autoinunune Syndrome Type Polyglandular, Beheet Syndrome, Celiac Disease, Chagas disease, Cholangitis, Sclerosing, Chronic Inflammatory Demyelinating Polyneuropathy, Chronic, lymphocytic thyroiditis, Churg-Strauss Syndrome, Colitis, Ulcerative, Crohn's disease, Cryoglobulinemia, Cushing Syndrome, Dermatitis Herpetiformis, Dermatomyositis, Diabetes Mellitus (Insulin-Dependent), Diffuse Cerebral Sclerosis of Schilder, Encephalomyelitis, Autoimmune, Experimental (EAE), Epidermolysis Bullosa Acquisita, Erythematosis, Felty's Syndrome, Glomerulonephritis (IGA), Glomerulonephritis Membranous, Goodpasture Syndrome, Graves' Disease, Guillain-Barre Syndrome, Hamman-Rich syndrome, Hepatitis Autoimmune, Hepatitis Chronic Active, Idiopathic thrombocytopenia, Inflammatory Bowel Diseases, Insulin resistance-type B, Lambert-Eaton. Myasthenic Syndrome, Lens-induced uveitis, Lichen Sclerosus et Atrophicus, Lupus Erythematosus Discoid, Lupus Erythematosus Systemic, Lupus Hepatitis, Lupus Nephritis, Lymphopenia, Meniere's Disease, Mixed Connective Tissue Disease, Mooren's ulcer, Mucocutaneous Lymph Node Syndrome, Multiple Sclerosis, Myasthenia Gravis, Transverse, Myocarditis, Narcolepsy, Neuritis Autoimmune Experimental, Neuromyelitis Optica, Oculovestibuloauditory syndrome, Ophthalmia Sympathetic, Opsoclonus-Myoclonus Syndrome, Pancreatitis, Pemphigoid Bullous, Pemphigus foliaceous, Pemphigus Vulgaris, Polyarteritis Nodosa, Polychondritis Relapsing, Polyendocinopathies Autoimmune, Polymyalgia Rheumatica, Polyradiculoneuropathy, Primary biliary cirrhosis, Psoriasis, Purpura Thrombocytopenic Idiopathic, Raynauds, Reiter Disease, Rheumatic Fever, Rheumatoid Arthritis, Sarcoidosis, Scleroderma, Sjögren's Syndrome, Spondylitis Ankylosing, Stiff-Person Syndrome, Still's Disease Adult Onset, Takayasu's Arteritis, Temporal Arteritis, Thyrotoxicosis, Type B Insulin Resistance, Uveomeningoencephalitic Syndrome, Wegener's Granulomatosis, Vitiligo. In addition diseases that can partly be involved with autoimmune reactivity are arteriosclerosis, Parkinsons disease, and Alzheimer's disease. A specific group of interesting diseases and disorders includes Diabetes Mellitus Type I Rheumatoid Arthritis, Systemic Lupus Erythematosus, Chronic lymphocytic thyroiditis, Multiple Sclerosis and Ulcerative Colitis.

Alternatively it may he used for the manufacturing of a medicament to be used in transplantations to inhibit immune rejection of organs, tissues, normal or gene therapeutically modified cells

### BRIEF DESCRIPTION OF THE DRAWINGS

FIG. 1 shows the tryptophan catabolism, EC 1.13.11.11 is tryptophan 2,3-dioxygenase, EC 1.13.11.52 indoleamine 2,3-dioxygenase (IDO), 1.14.13.9 is kynurenine 3-monooxygenase, EC 2.6.1.7 is kynurenineoxoglatarate transaminase, EC 3.5.1.9 is arylformamidase and EC 3.7.1.3 is kynureninase.

FIG. 2 shows the chemical structure of zebularine

FIG. 3 shows qRT-PCR analysis of the IDO mRNA expression levels in H1D2WT, H1D2IL12C46, and H1D2IL18C2 rat colon cancer cell lines untreated or treated with zebularine at 20 μM and 100 μM of zebularine respectively. The values given are normalized in relation to the HPRT expression values.

FIG. 4 shows zebularine treated lymphocytes. FIG. 4A show proliferation of stimulated human lymphocytes after five days in culture in the presence of zebularine at different concentrations, FIG. 4B show restoration of inhibited proliferation of stimulated human lymphocytes after five days in culture in the presence of zebularine at two different concentrations and the IDO inhibitor 1-methyl tryptophane at 100 μM and FIG. 4C show proliferation of stimulated rat spleen lymphocytes after four days in culture in the presence of zebularine at different concentrations.

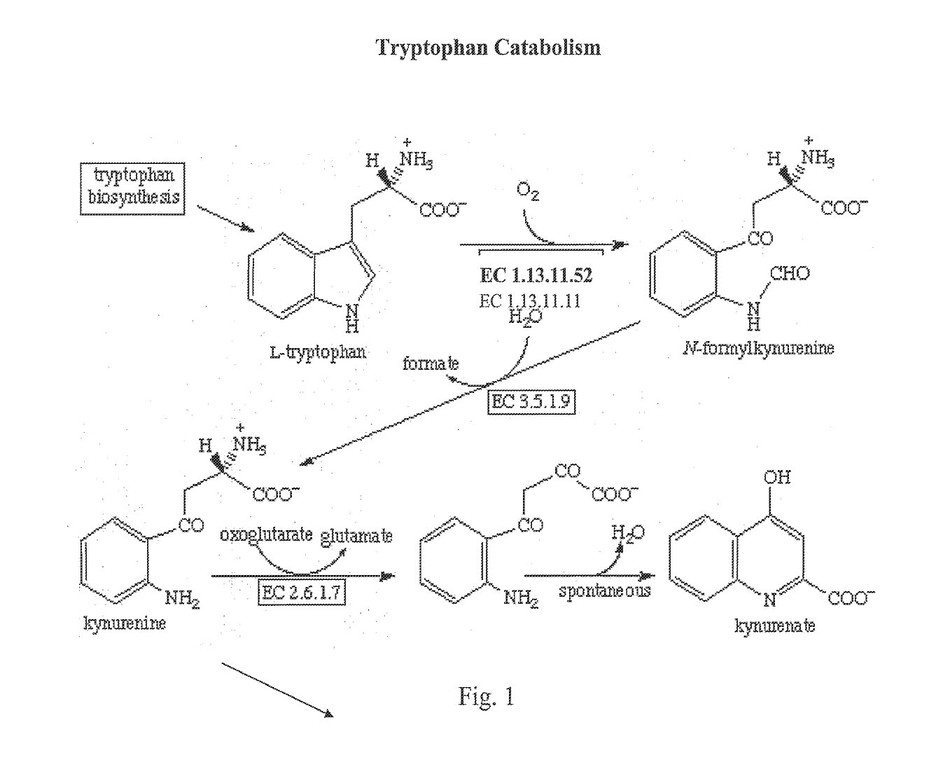

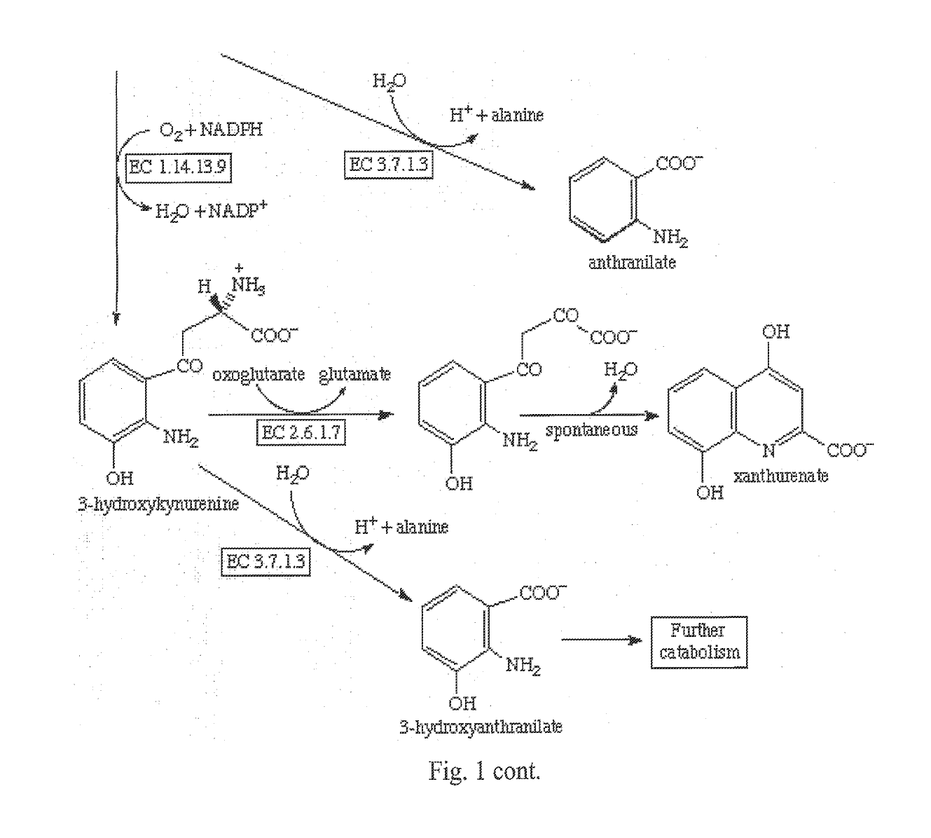

### DETAILED DESCRIPTION OF THE INVENTION

In the context of the present application and invention, the following definitions apply:

The term “immunoprotective” is defined herein as an effect which reduces, arrests, or ameliorates immunological insult and is protective, resuscitative or revivative for affected tissue that has suffered cytotoxic insult, from immune cells or inflammation.

The term “immunoprotective agent” is herein defined as active ingredient or medicament containing an immune insult treatment dose of active ingredient effective in reducing, preventing, arresting, or ameliorating immune insult and provides protection, resuscitation or revival to affected tissue that has suffered immune mediated insult.

The term “indolamine dioxygenase (IDO)” is intended to mean IDO-1 (indoleamine 2,3-dioxygenase, EC 1.13.11.52), IDO-2 (incloleamine-pyrrole 2,3 dioxygenase-like 1, EC 1.13.11.-) or TDO (tryptophan 2,3-dioxygenase, EC 1.13.11.11) that are three different proteins that can catabolize tryptophan. IDO-1 can also catabolize serotonin and melatonin although the substrate specificity for IDO-2 and TDO is not so well studied. Metabolites or catabolites from the tryptophan pathway are Tryptophan, N-Formyl-kynurenine, Formylanthranilate, Anthranilate, L-Kynurenine, 4-(2-Aminophenyl)-2,4-dioxybutartoate, Kyrturenic acid, 3-Hydroxy-L-kynurenine, 3-Hydroxy-anthranilate, 3-Metoxy-anthranilate, 4-(2-Amino-3-hydroxy-phenyl)-2,4-dioxobutanoate, Xanthurenate, 8-Metoxy-kurenate, 2-Amino-3-carboxy-muconate semialdehyde, 2-Aminomuconate semialdehyde, Quimolinic acid, Cinnavalininate, Tryptamine, N-Methyltryptamine, Indoleacetate, 2-Formamino-benzoylacetate, 5-Hydroxy-L-tryptophan, 5-Hydroxy-N-formylkunerine, 5-Hdroxy-kunerine, 5-Hydroxy-kunerenamin, 4,6-Dihydroxy-quinoline, Serotonin, N-Acetyl-serotonin, Melatonin, 6-Hydroxy-melatonin, Formyl-N-acetyl-5-metoxykynurenamine, N-Methylserotonin, Formyl-5-hydroxy-kynurenamine, 5-Metoxytryptamine, 5-Hydroxyindole-acetaldehyde, 5-Hydroxyindoleacetate, 5-Metoxyindoleacetate, 5-Hydroxyindole-acetylglycine to enhance the immunosuppressive IDO activity. Examples are Kynurenine, 3-hydroxy-kynurenine, arithranilic acid, 3-hydroxy-anthranilic acid, quinolinic acid and picolinic acid. Also enhancements using synthetic variants of tryptophan catabolites, e.g., N-(3,4,-Dimethoxycinnamoyl) anthranilic acid. The immune suppression mediated by IDO is mediated by starvation of Tryptophan, induction of apoptosis in lymphocytes and induction of regulatory T lymphocytes (Treg). Hence, the apoptosis induction and Tres induction is mediated by the catabolites, why addition of such catabolites in combination with IDO induction by medicament of the invention may enhance the clinical effect. The immune suppressive action from IDO-1, IDO-2 and TDO may be explained by 1) starvation of tryptophan, 2) direct toxic effect from several of die above mentioned metabolites/catabolites that induce apoptosis of immune cells, particularly L-Kynurenine, Anthranilate, 3-Hydroxy-anthranilate and 3-Hydroxy-L-kynurenine and 3) that some of the metabolites/catabolites stimulate the differentiation of T helper cells to immune suppressive regulatory T-cells important for tolerance.

An analogue is a molecule that differs in chemical structure from a parent compound, for example a homolog (differing by an increment in the chemical structure, such as a difference in the length of an alkyl chain), a molecular fragment, a structure that differs by one or more functional groups, a change in ionization. Structural analogues are often found using quantitative structure activity relationships (QSAR), with techniques such as those disclosed in Remington (The Science, and Practice of Pharmacology, 19 Edition (1995), chapter 28). A derivative is a substance related to a base structure, and theoretically derivable from the base structure. A mimetic is a biomolecule that mimics the activity of another biologically active molecule. Biologically active molecules can include chemical structures that mimic the biological activities of a compound, for instance zebularine.

The IDO gene expression is known to be induced in antigen presenting cells and is subject to complex regulation by an array of signals. For example, IFN-γ can signal through JAK and STATI together with the sis-acting IFN-γ-stimulated response elements (ISRE) on the IDO promoter, activating transcription of IDO. However, bacterial lipopolysaccharides (LPS), interleukin-1-beta (IL-1β), and TNF can also enhance IDO expression. Also in human epithelial cells, IFN-γ-induced IDO expression is transcriptionally enhanced by tumour necrosis factor-alpha (TNF-α). It is possible that also an IFN-γindependent induction mechanism exists.

The invention relates to the use of at least 1-(β-D-Ribofuranosyl)-1,2-dihydropyrimidin-2-one, analogue, derivative or mimetic or salts thereof for the manufacturing of a medicament for the treatment of an autoimmune disorder or disease or immune rejection of transplants or gene therapeutically modified cells, wherein the treatment induces indolamine dioxygenase. Said composition will be used as an immunoprotective agent. Examples of diseases are listed above.

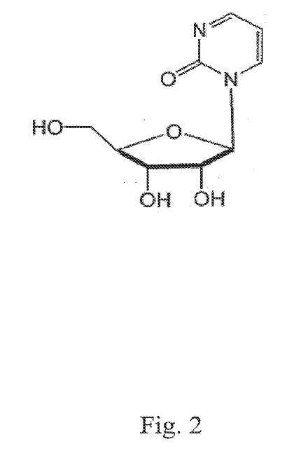

1-(β-D-Ribofuranosyl)-1,2-dihydropyrimidin-2-one also named Zebularine (FIG. 2) is a drug that the inventors have shown to strongly enhance the production of IDO in concentrations of 100 μM, which is far below toxicity level.

Zebularine will induce the production of IDO that is a natural immune suppressive molecule. As such, zebularine would help to control unwanted immune reactivity. Moreover it is likely that induction of IDO can lead to the induction of tolerance that would make continuous immunosuppressive therapy unnecessary and in this way avoid associated side effects.

Another name of zebularine as 2′-t-Butyldimethylsilyl-3′-O-[(di-isopropylamino)(2-cyanoethoxy)phosphino]-5′-O-4,4′-dimethoxytrityl)-2(1H)-pyrimidinone-1-β-D-riboside, zebularine, is a useful and effective inducer of immune suppression and inducer of the IDO production. The chemical structure of Zebularine is shown in FIG. 2.

Zebularine Synonyms are 1-(β-D-Ribofuranosyl)-1,2-dihydropyrimidin-2-one or 2-Pyrimidone-1-β-D-riboside. Examples of analogues, mimetics and derivatives that may be used include but are not limited to 5-methylcytidine, 2′-deoxyzebularine, 5-fluoro-zebularine, 5-fluoro-2′-dexyzebularine, 5-chloro-zebularine, 5-chloro-2′-dexyzebularine, 5-bromo-zebularine 5-bromo-2′-dexyzebularine, 5-iodo-zebularine, 5-iodo-2′-dexyzebularine, 5-methylpyrimidin-2-one. 5-Me-2′-deoxyzebularine, or mono, di or tri phosphates thereof

The present invention is further that the invented medicament such as zebularine containing an immunoprotective treatment dose of active ingredients aimed as an agents in situations where autoimmune reactivity is involved in the central nervous system, such as the diseases mentioned above.

Zebularine represents a completely new class of substances for use as immunomodulators for immunosuppression and tolerance induction. Zebularine an immunoprotective agent at acceptable physiologic and pharmacologic doses.

The invention is the use of for example zebularine for obtaining a treatment medicine and medicament, intended for the therapeutic immunoprotective use of treating autoimmune disease and patients receiving allotransplants or undergoing gene therapy with associated risks of rejection of cells expressing the transferred genes. Examples of other diseases are found above.

The medicament, such as zebularine is suited for the immunoprotectiye treatment of cell cultures, in vitro treatment of transplants, transfected, genetically engineered cell cultures or transplants.

The medicament may be administrated to a mammal in need thereof in a suitable amount to achieve an effect corresponding to such concentrations that induce a strong activity in vitro of said methyl transferase inhibitor alone or in combination with other inducers of IDO. Other inducers of IDO can be free fatty acids, interferons, toll-like receptor ligands, histone deactylase inhibitors (HDACi).

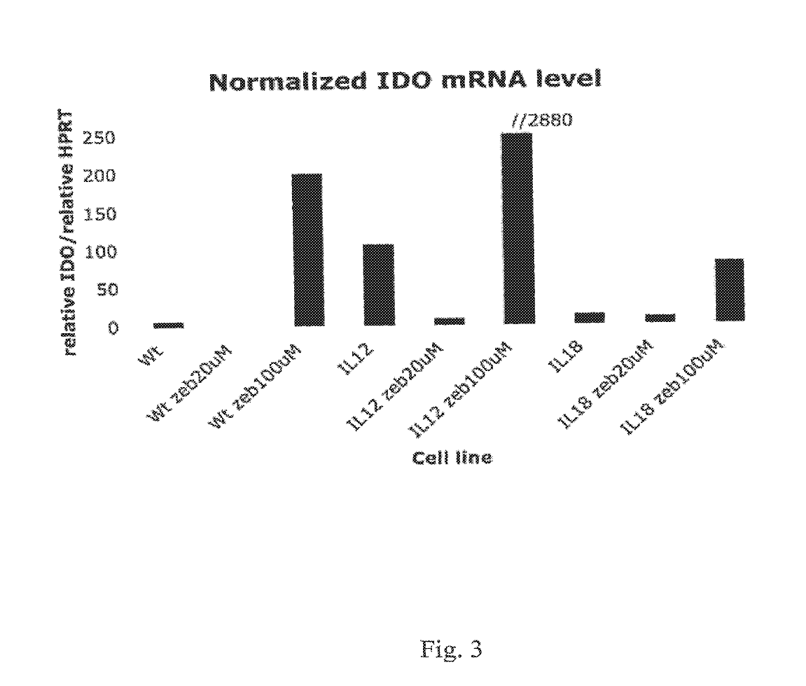

The inventors investigated how zebularine, a methyl transferase inhibitor, affected the IDO production from three rat colon cancer cell lines (FIG. 3), and found that the IDO expression dramatically increased at a zebularine concentration of 100 μM. The treatment with 100 μM of zebularine induced IDO mRNA expression in all three cell lines. A 48-fold increase of the IDO mRNA level in the H1D2WT cell line, a 24-fold increase in the H1D2IL12C46 cell line and a 14-fold increase in the H1D2IL18C2 cell line were detected. Immunization of rats with tumour cells pretreated with 100 μM of zebularine also resulted in a very weak proliferative response of non-adherent spleen cells stimulated with tumor cells in vitro. The inventors know that the 100 μM treatment of tumour cells strongly induces the production of the immunosuppressive molecule IDO but the induction of other T cell immunosuppressive molecules might also be involved, although no evidence has been found for an effect on prostaglandine E2 (PGE2) or nitric oxide (NO) production. It seems that the 100 μM zebularine treatments are not affecting the PGE2 production, the NO production, or the production of tumour antigens.

By having a direct effect on the immune system, treatment with a dose corresponding to an in vitro dose of 5 to 1000 μM zebularine, alone or in combination with other inducers of IDO such as free fatty acids, interferons, toll-like receptor ligands or histone deactylase inhibitors (HDACi) can be used for pretreatment of transplants (organs, tissues or cells) inducing IDO expression in the endothelial cells and as a consequence making them less immunogenic to the host and reducing the risk of rejection of the grafted cells. By subsequent treatment of the graft recipients with zebularine at a dose providing immune suppression in vivo and induction of immunological tolerance permanent survival of the transplants can be achieved without further therapy or with minimal such therapy. Similar treatment can be used to control and even cute autoimmume diseases, such as arthritis, MS and diabetes type I by inducing tolerance to the tissue specific antigens involved.

Zebularine is highly hydrophilic, and soluble in water. Zebularine may be administered orally or intravenously (i.v.) or by any other suitable route. In the mouse, the bioavailability after i.v. injection is good and about 60% of a dose of 100 mg/kg is absorbed in tissues like plasma, red blood cells, liver, kidney, spleen, heart, lung, muscle, Duodenum, Jejunum, Ileum, Colon and Carcass. The bioavailability for Fat, Brain, Testes, Cecum and Stomach is less. The peak concentrations are reached 5 to 10 minutes after administration and the turnover is quite fast for many tissues. The bioavailability has been compared to that of uridine and found to be very similar.

Zebularine in the mouse mainly metabolized to uridine, uracil and dihydrouracil.

When a patient is to be treated by the invented medicament such as zebularine it may be administrated in an amount which varies between 5 um to 200 uM, depending on which disease or disorder to be treated. Initially a higher dose may be used such as 75 to 1000 uM or 100 uM followed by a maintaining dose of 5-65 uM, such as 50 uM.

The invented medicament may further comprise a pharmaceutically acceptable buffer, excipient, diluent or carrier.

“Pharmaceutically acceptable” means a non-toxic material that does not decrease the effectiveness of the biological activity of the active ingredients. Such pharmaceutically acceptable buffers, carriers or excipients are well-known in the art (see Remington's Pharmaceutical Sciences, 18th edition, A. R Gennaro, Ed., Mack Publishing Company (1990) and handbook of Pharmaceutical Excipients, 3rd edition, A, Kibbe, Ed. Pharmaceutical Press (2000).

The term “buffer” is intended to mean an aqueous solution containing an acid-base mixture with the purpose of stabilising pH. Examples of buffers are Trizma, Bicine, Tricine, MOPS, MOPSO, MOBS, Tris, Hepes, HEPBS, MES, phosphate, carbonate, acetate, citrate, glycolate, lactate, borate, ACES, ADA, tartrate, AMP, AMPD, AMPSO, BES, CABS, cacodylate, CHES, DIPSO, EPPS, ethanolamine, glycine, HEPPSO, imidazole, imidazolelactic acid, PIPES, SSC, SSPE, POPSO, TAPS, TABS, TAPSO and TES.

The term “diluent” is intended to mean an aqueous or non-aqueous solution with the purpose of diluting the medicament. The diluent may be one or more of saline, water, polyethylene glycol, propylene glycol, ethanol or oils (such as safflower oil, corn oil, peanut oil cottonseed oil or sesame oil). The excipient may be one or more of carbohydrates, polymers, lipids and minerals. Examples of carbohydrates include lactose, sucrose, mannitol, and cyclodextrines, which are added to the composition, for facilitating lyophilisation. Examples of polymers are starch, cellulose ethers, cellulose carboxymethylcellulose, hydroxypropylmethyl cellulose, hydroxyethyl cellulose, ethylhydroxyethyl cellulose, alginates, carageenans, hyaluronic acid and derivatives thereof, polyacrylic acid, polysulphonate polyethylenglycol/polyethylene oxide, polyethyleneoxide/polypropylene oxide copolymers, polyvinylalcohol/polyvinylacetate of different degree of hydrolysis, and polyvinylpyrrolidone, all of different molecular weight, which are added to the composition, e.g., for viscosity control, for achieving bioadhesion, or for protecting the lipid from chemical and proteolytic degradation. Examples of lipids are fatty acids, phospholipids, mono-, di-, and triglycerides, ceramides, sphingolipids and glycolipids, all of different acyl chain length and saturation, egg lecithin, soy lecithin, hydrogenated egg and soy lecithin, which are added to the composition for reasons similar to those for polymers. Examples of minerals are talc, magnesium oxide, zinc oxide and titanium oxide, which are added to the composition to obtain benefits such as reduction of liquid accumulation or advantageous pigment properties.

The invented medicament may administrated by any suitable route including oral, sublingual, buccal, nasal, inhalation, parenteral (including intraperitoneal, intraorgan, subcutaneous, intradermal, intramuscular, intra-articular, venous (central, hepatic or peripheral), lymphatic, cardiac, arterial, including selective or superselective cerebral arterial approach, retrograde perfusion through cerebral venous system, via catheter into the brain parenchyma or ventricles), direct exposure or under pressure onto or through the brain or spinal tissue, or any of the cerebrospinal fluid ventricles, injections into the subarachnoid, brain cisternal, subdural or epidural spaces, via brain cisterns or lumbar puncture, intra and periocular instillation including application by injection around the eye, within the eyeball, its structures and layers, the ear, including the Eustachian tube, mastoid air cells, external and internal auditory canals, tympanic membrane, middle ear, inner ear including the cochlear spiral ganglion and labyrinthine organs, as well as via enteral, bowel, rectal, vaginal, urethral or bladder cisternal. Also for in utero and perinatal indications then injections into the maternal vasculature, or through or into maternal organs including the uterus, cervix and vagina, and into embryo, foetus, neonate and allied tissues and spaces such as the amniotic sac, the umbilical cord, the umbilical artery or veins and the placenta, with parenteral being the preferred route. The preferred route may vary depending on the condition of the patient.

The effect of the invented medicament may be further potentiated not only by combining it with other IDO stimulating drugs as mentioned above but also in combination with an immunosuppressive agent to reduce the frequency of effector immune cells during or before the stimulation of tolerance.

This invention includes the possibility of the timing and sequence of delivery of active ingredients to induce tolerance, in order to have the best opportunity to protect tissue from immune mediated insult, the medicament needs to be available as soon as possible within the cells of the graft. This would induce IDO expression in endothelial cells, which by itself suppresses rejection responses.

The medicament may comprise additional active ingredients such as glycocorticoids, methotrexate, rapamycin, cyclophosphamide, antimetabolites including azathioprine, immunophilin-binding drugs (including tolerance, tacrolimus, sirolimus, everolimus), inhibitors of nucleotide synthesis (including mycophenolate mofetil, mizoribine, leflunomide, FK778), FIY720, lymphocyte depleting antibodies (including polyclonal antibodies to lymphocytes, thymocytes, T-cells, muromonab-CD3, rituximab, Alemtuzumab, CAMPATH-1), non-depleting antibodies (including daclizumab, basiliximab, the two CTLA-4-Ig fusion proteins LEA29Y and abatacept, LFA3-Ig fusion protein), anti-TNF antibodies (including infliximab, adalimumab), natalizumab (anti-VLA-4), the anti-CD154 antibodies BG9588 and IDEC 131), soluble cytokine receptors (including lenercept and etanercept (soluble TNF p55 and TNF p75 receptors), histone deacetylase inhibitors and anakinra (soluble IL-IRA).

Other examples includes transfer of genes such as arginase-1, or substances like prostaglandin E2 (PGE2), cyclosporine A.

These immune suppressive drugs mentioned above can be used in combination with the medicament of the invention to reduce the number of immune cells.

The bioavailability of the medicament such as zebularine is partly dependent on the activity of aldehyde oxidase that can add water to the position 4 of zebularine and thereby converting zebularine into uridine. The activity of aldehyde oxidase is high in the liver. There exist a large number of aldehyde oxidase inhibitors (see Obach et al, 2004) and inhibitors with IC₅₀ values from 3 nM up to 1 μM are e.g., Raloxifene, Perphenazine, Thioridazine, Menadione, Trifluperazine, Amitriptyline, Estradiol, Felodipine, Clomipramine, Loratidine, Promethazine, Chlorpromazine, Ethinyl estradiol, Norclomipramine, Amodiaquine, Nortriptylin.

The medicament includes those suitable for administration by the routes including oral, sublingual, buccal, nasal, inhalation, parenteral (including intraperitoneal, intraorgan, subcutaneous, intradermal, intramuscular, intra-articular, venous (central, hepatic or peripheral), lymphatic, cardiac, arterial, including selective or superselective cerebral arterial approach, retrograde perfusion through cerebral venous system, via catheter into the brain parenchyma or ventricles), direct exposure or under pressure onto or through the brain or spinal tissue, or any of the cerebrospinal fluid ventricles, injections into the subarachnoid, brain cisternal, subdural or epidural spaces, via brain cisterns or lumbar puncture, intra and periocular instillation including application by injection around the eye, within the eyeball, its structures and layers, the ear, including the Eustachian tube, mastoid air cells, external and internal auditory canals, tympanic membrane, middle ear, inner ear including the cochlear spiral ganglion and labyrinthine organs, as well as via enteral, bowel, rectal, vaginal, urethral or bladder cisternal. Also for in utero and perinatal indications then injections into the maternal vasculature, or through or into maternal organs including the uterus, cervix and vagina, and into embryo, fetus, neonate and allied tissues and spaces such as the anmiotic sac, the umbilical cord, the umbilical artery or veins and the placenta, with parenteral being the preferred route.

The medicament may be distributed and made available in convenient unit dose form such as capsules and ampoules, containing the active ingredient of the invention, and may be manufactured and distributed by any of the methods known to the pharmaceutical arts. In addition to the active ingredient, the medicament can also contain other usual agents of the art relating to the type of medicament produced. This may, by example, take the configuration of suspensions, solutions and emulsions of the active ingredient in lipid, non-aqueous or aqueous diluents. solvents, dissolving agents, emulsifiers, syrups, granulates or powders, or mixtures of these. The medicament can also contain colouring agents, preservatives, perfumes, flavouring additions and sweetening agents. In addition to the active ingredient, the medicament can also contain other pharmaceutically active medications. The manufacture and distribution of the medicament is carried out by techniques known to the art, such as, evenly and intimately bringing together the active ingredient with liquids or fine solids or both, and then if needed, forming the medicament into a dose unit form. The discrete dose, portion and carrier vehicle constituting the medicament will generally be adapted by virtue of shape or packaging for medical administration and distributed for this purpose.

Tablets can be manufactured and distributed by compression or mould, from active ingredient possibly with one or more additional pharmaceutically active compounds. Compressed tablets can be manufactured and distributed through compression in a machine typical to the art a known quantity of the active ingredient in a dispersible configuration such as powder or granules, possibly mixed with other agents including binders, lubricants, inert diluents preservatives, and dispersing agents. Moulded tablets can be manufactured and distributed by moulding in a machine typical to the art a mix of known quantity of active ingredient addition pharmaceutically active compounds and other additives moistened with a liquid diluent. The tablets can possibly be coated, enveloped or covered, with substances incinding protective matrices, which can contain opacifiers or sweeteners and can be formulated to allow slow or controlled release, or also release within a certain part of the digestive system of the contained active ingredients. Capsules can be manufactured and distributed by placement of a known quantity of active ingredient, additional pharmaceutically active compounds and additives within a two part or sealed capsule of gelatine or other aqueous dissolvable substance. The active ingredient can also be manufactured and distributed as a medicament in microencapsulated, microsomal, micellar and microemulsion forms.

The medicament containing the active ingredient acceptable for oral topical administration can be manufactured and distributed as lozenges containing the active ingredients, other pharmaceutically active compounds, and additives in a flavoured basis, such as acacia and tragacanth; Pastilles containing the active ingredient with other pharmaceutically active compounds, and additives in an inert base such as gelatine and sucrose: Mouthwashes or rinses containing the active ingredient with other pharmaceutically active compounds, and additives in an acceptable liquid.

The medicament containing the active ingredient acceptable for skin topical administration can be manufactured and distributed as ointments, oils, creams, lotions, gels, pastes and transdermal patch containing the active ingredient, other pharmaceutically active compounds, additives and an acceptable carrier medium.

The medicament containing the active ingredient acceptable for nasal administration can be manufactured and distributed with other pharmaceutically active compounds and additives as a powder for inhalation, or as an oily, aqueous or non-aqueous liquid for nasal spray or drops.

The medicament containing the active ingredient acceptable for rectal administration can be manufactured and distributed as suppositories, creams, foams, douches or enemas with other pharmaceutically active compounds, suitable bases of the usual water-soluble diluents, fats, and additives known to practitioners of the art.

The medicament containing the active ingredient acceptable for vaginal administration can be manufactured and distributed as pessaries, suppositories, creams, gels, foams, douches or sprays with other pharmaceutically active compounds, suitable bases and additives known to practitioners of the art.

The medicament containing the active ingredient acceptable for parenteral administration can be manufactured and distributed from aqueous and non-aqueous sterile injection solutions, other pharmaceutically active compounds, additives including anti-oxidants, bacteriostats and solutes and sugars such as mannitol to make the medicament isotonic, hypotonic or hypertonic with the blood of the recipient; and also aqueous and non-aqueous sterile suspensions which can include suspenders and thickeners. The medicament can be manufactured and distributed in unit-dose or mild-dose containers, such as sealed glass or plastic ampoules, vials, bottles and bags as a liquid, and in a dry state requiring only the addition of sterile liquid, for example water, saline or dextrose solutions, immediately prior to use. Extemporaneous solutions and suspensions for injection can be prepared from powders and tablets of the kind above described.

The medicament containing the active ingredient acceptable for administration into the brain and related structures, spinal cord and related structures, ventricular system and cerebrospinal fluid spaces can be manufactured and distributed from aqueous and non-aqueous sterile injection solutions, other pharmaceutically active compounds, additives including anti-oxidants, bacteriostats and solutes and sugars such as mannitol to make the medicament isotonic, hypotonic or hypertonic with the cerebrospinal fluid, and also aqueous and non-aqueous sterile suspensions which can include suspenders and thickeners. The medicament can be manufactured and distributed in unit-dose or multi-dose containers, such as sealed glass or plastic ampoules, vials, bottles and bags as a liquid, and in a dry state requiring only the addition of sterile for example water, saline or dextrose solutions, immediately prior to use. Extemporaneous solutions and suspensions for injection can be prepared from powders and tablets of the kind above described.

The desired unit dose of medicaments, are those containing a daily dose or immune insult treatment dose or an appropriate fraction thereof, of the administered active ingredient. Unit dose forms of the invention may also include more complex systems such as double barrelled syringes, syringes with sequential compartments one of which may contain the active ingredient, and the other any necessary diluents or vehicles. The agents in the syringes would be released sequentially or as a mixture or combination of the two after the triggering of the syringe plunger. Such systems are known in the art.

The medicament may be used for the treatment of a disease or disorder such as those mentioned above.

Following examples are intended to illustrate, but not to limit the invention in any manner, shape, or form, either explicitly or implicitly.

### EXAMPLES

Example 1: Organs prepared for transplantation was perfused with cold physiological salt saline (or sodium chloride) solution containing Zebularine at a concentration of 100 μM and incubated in that solution until the surgical transplantation procedure was performed. One day prior to transplantation, the recipient patient is treated with oral Zebularine at an optimal dose for induction of IDO at an immunosuppressive concentration and oral cyclosporine at standard dosage continued daily for 2 weeks and at a reduced dose level for another 2 weeks. During the 4 first days after surgery dexamethasone is administered intravenously at a daily dose of 8 mg and the dose is then tapered over the following 2 weeks. One month after transplantation cyclosporine treatment is stopped, whereas the Zebularine is continued and patients are closely watched for evidence of rejection. After 2 months of treatment with Zebularine alone this treatment is stopped provided nonresponsiveness to donor but not third part cells can be demonstrated in vitro.

Example 2: A patient with a bilateral severe ocular-surface disorder is to receive allogeneic corneal epithelial stem-cell transplantation (as reported by Tsubota et al, NEJM, 340, 1697-1703, 1999). One day prior to transplantation the recipient patient is treated with oral Zebularine at an optimal dose for induction of IDO at an immunosuppressive concentration and oral cyclosporine at standard dosage continued daily for 2 weeks and at a reduced dose level for another 2 weeks. During the 4 first days after surgery dexamethasone is administered intravenously at a daily dose of 8 mg and the dose is then tapered over the following 2 weeks. Chic month after transplantation cyclosporine treatment is stopped, whereas the Zebularine continued and patients closely watched for evidence of rejection. After 2 months of treatment with Zebularine alone this treatment is stopped provided nonresponsiveness to donor but not third part cells can be demonstrated in vitro.

Example 3: A patient suffering from critical limb ischemia is subjected to gene therapy with direct intramuscular administration of the eukaryotic expression vector pUC18 encoding VEGF₁₆₅ transcriptionally regulated by the cytomegalovirus promoter/enhancer in a total of 4 mg of DNA in 8 aliquots into the ischemic limb as previously described (Kalka et al., CIRC RES 2000; 86:1198-1202). The day before gene transfer the patient receives oral Zebularine at an optimal dose for induction of IDO at an immunosuppressive concentration and this treatment is continued for 3 months by oral administration in order to prolong the action of the VEGF protecting against the immune rejection of the muscle cells expressing the vector-encoded proteins, Plasma VEGF levels are measured by an ELISA assay to monitor the maintained expression of the transferred VEGF gene.

Example 4: A patient with active Rheumatoid Arthritis with considerable remaining symptoms despite treatment with Methotrexate 10 mg per week receives oral Zebularine at an optimal dose for induction of IDO at an immunosuppressive concentration. Methotrexate therapy is continued for 6 weeks. Zebularine them then continued as single therapy for 3 months in order to induce immunosuppression and immunological tolerance.

Example 5: A patient with a diagnosis of relapsing-remitting multiple sclerosis, a score on the Expanded Disability Status Scale of 4 (on a range of 0-10), a MRI scan revealing lesions consistent with a diagnosis of multiple sclerosis, and having had a relapse despite having received treatment with interferon beta-1a for more than 12 months at a dose of 30 microgram i.m. per week. At maintained interferon beta-1a therapy oral Zebularine therapy is initiated at an optimal dose for induction of IDO at an immunosuppressive concentration. The interferon beta-1a therapy is stopped after 3 weeks and Zebularine therapy continued for 12 months and then stopped provided that no relapse has occurred.

Example 6: A young patient with the diagnosis Diabetes Mellitus type I in an early stage is treated with insulin and within 3 of diagnosis with oral Zebularine at an optimal dose for induction of IDO at an immunosuppressive concentration. The required insulin dose is closely monitored beyond the expected initial decrease until it has stabilized at a low level or is no longer required. Zebularine therapy is maintained for 4 months and then stopped. Resumed Zebularine therapy dependent on a close monitoring of signs of increased requirement of insulin.

Example 7: Immune cells treated with zebularine in vitro (The use of in vitro treatment of immune cells with zebularine) can be utilized for transplantation. Peripheral blood from a patient with diabetes type I who is to receive a (that will be given) pancreatic beta cell transplant (transplantation) is collected. White blood cells from the patient blood are isolated and cultured in the presence of GMCSF, protein extract from the donor and zebularine. The cells are then stimulated with CpG-containing oligonucleotides and after farther culture in vitro in presence of zebularine and HDAC inhibitors, regulatory plasmacytoid dendritic cells are enriched and are given back to the patient. (The in vitro treatment can also be combined with e.g. treatment with CTLA4-Ig or HDAC inhibitors.) These regulatory DC will then present antigens from the donor and induce tolerance against these foreign antigens. The beta cells from the donor are treated in vitro with zebularine prior to tranplantation. The patient can be given a pretreatment with zebularine, alone or in combination with HDAC-inhibitors, rapamycin, CTLA4-Ig, alone or in combination). The in vivo treatment will continue for about 2 weeks after transplantation of the pancreatic, insulin producing beta-cells. The patient is monitored by FACS analysis for presence of FoxP3 regulatory T-cells having T-cell receptors that can bind beta cell peptides conjugated with soluble fluorescent HLA-DR complexes. (The analysis is followed using a fluorescence activated cell sorter (FACS).) When an increased frequency (frequence) of regulatory T-cells can be identified, the immune suppressive treatment will be reduced until the patient does not need immune suppression and is cured.

Example 8: Immune cells treated with zebularine in vitro (The use of in vitro treatment of immune cells with zebularine) can be utilized in patients with autoimmune disease (for autoimmunity). Peripheral blood from a patient with reumatoid arthritis is collected. White blood cells from the patient blood are isolated and cultured in the presence of GMCSF, collagen or cartilage and zebularine. The cells are then stimulated with CpG-containing oligonucleotides and after further culture in vitro in presence of zebularine and HDAC inhibitors, regulatory plasmacytoid dendritic cells are enriched and are given back to the patient. (The in vitro treatment can also be combined with e.g., treatment with CTLA4-Ig or HDAC inhibitors.) These regulatory DC will then present antigens from collagen and reinduce (induce) tolerance against these self antigens (that is broken) in the RA patient. The patient can be given a pretreatment with zebularine, alone or in combination with HDAC-inhibitors, rapamycin, CTLA4-Ig (alone or in combination). In vivo treatment will continue for about 2 weeks after injection of the in vitro treated tolerogenic DC cells. The patient is monitored by FACS analysis for presence of FoxP3 regulatory T-cells having T-cell receptors that can bind collagen peptides conjugated with soluble fluorescent HLA-DR complexes. The patient is also monitored for the presence of CD8+ effector T-cells with TcR that can bind soluble fluorescent HLA-A proteins conjugated with collagen peptides. (The analysis is followed using a fluorescence activated cell sorter (FACS).) When an increased frequency (frequence) of regulatory T-cells and a significant decrease in CD8+ effector cells can be identified, the immune suppressive treatment will be reduced until the patient does not need immune suppression and is cured, i.e., the natural tolerance has been restored.

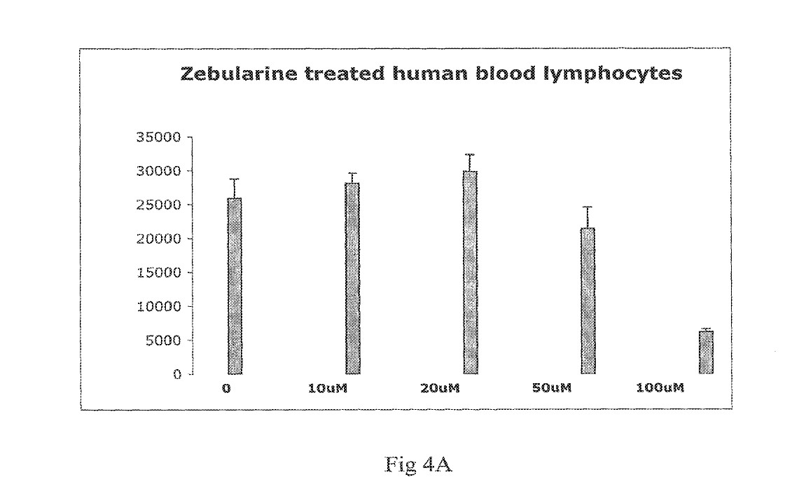

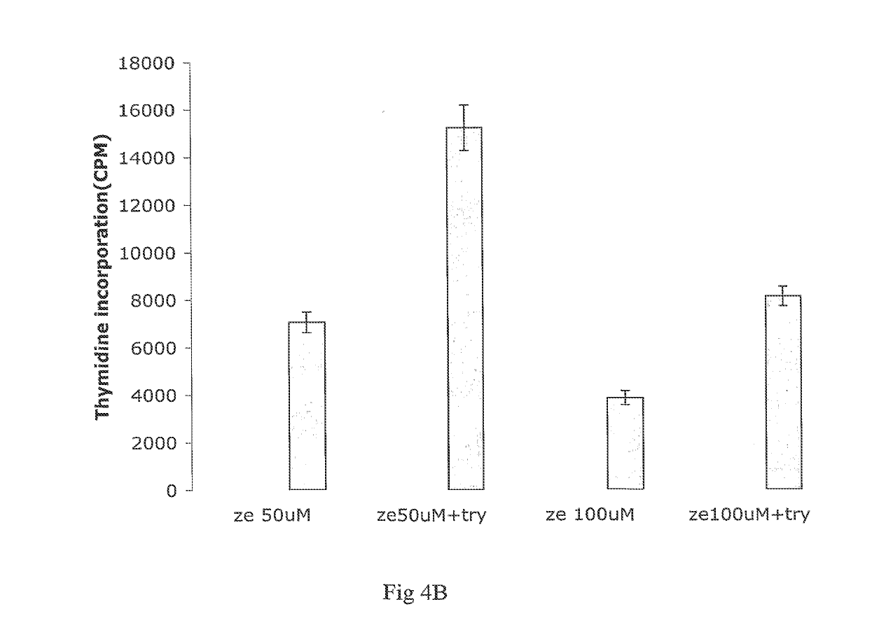

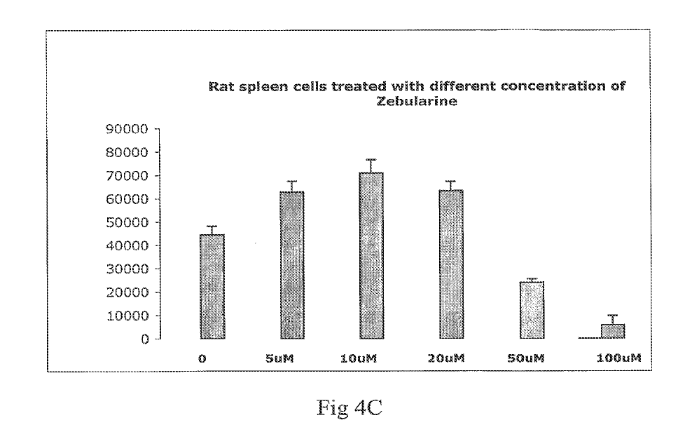

Example 9: In an experiment the inventors tested the immunogenicity of genetically engineered rat colon cancer cells expressing rat interleukin 12. These cancer cells have repeatedly been used to immunize rats that create a strong immune response towards the rat tumour cells. The inventors pre-treated these rat colon cancer cells with 100 μM zebularine that rendered the rat colon cancer cells less immunogenic when used for immunization. This was congruent with the changes in expression of IDO (FIG. 3). Similar treatments of rat spleen cells and human buffy coat leukocytes induce enhanced IDO production and strong suppression of polyclonal lymphocyte proliferation, i.e. suppression of T cell activation. There is a very reproducible induction of IDO (about 20-fold) from different human leukocyte cultures with a slight interindividual variation. The suppression of proliferation can be blocked by addition of the IDO inhibitor 1-methyl tryptophan (1-MT)as can be seen from FIG. 4.

References

Cheng, J. C., C. B. Matsen, F. A, Gonzales, W. Ye, S. Greer, V. E. Marquez, et al., Inhibition of DNA methylation and reactivation of silenced genes by Zebularineularine. Journal of the National Cancer Institute 95 (2003)199-409.

Cheng, J. C., DS. J. Weisenberger, F. A. Gonzales, G. Liang, G. L. Xu, Y. G. Hu, et al., Continuous Zebularineularine treatment effectively sustains demethylation human bladder cancer cells. Molecular and Cellular Biology 24 (2004) 1270-1278.

Cheng, J. C., C. B. Yoo, D. J. Weisenberger, J. Chuang, C. Wozniak, G. Liang, et al., Preferential response of cancer cells to Zebularineularine, Cancer Cell 6 (2004) 151-158.

Liu, G., H. Ying, G. Zeng, C. J. Wheeler, K. L. Black, J. S. Yu, HER-2, gp100, and MAGE-1 are expressed in human glioblastoma and recognized by cytotoxic T cells, Cancer Research, 64 (2004) 4980-4986.

Munn, D. H., M. D. Sharma, D. Hou, B. Baban, J. R. Lee, S. J. Antonia, et al., Expression of indoleamine 2,3-dioxygenase by plasmacytoid dendritic cells in draining lymph nodes. The journal of Clinical Investigation, 114 (2004) 280-290.

Obach R. S., Huynh P., Allen M. C. and Beecham C. Human Liver Aldehyde Oxidase: Inhibition by 239 Drugs, Journal of Clinical Pharmacology, 2004 44;7-19

Miki T, Sun H, Lee Y, Tandin A, Kovscek A M, Subbotin V, Fung J J, Valdivia L A. Blockade of tryptophan catabolism prevents spontaneous tolerogenicity of liver allografts. 2001 Transplantation Proceedings, Volume 33, Issue 1-2, Pages 129-130

Mellor, A. L., D. H. Munn, IDO expression by dendritic cells: tolerance and tryptophan catabolism, Nature Reviews Immunology 4 (2004) 762-774.

Uyttenhove, C. L. Pilotte, I. Theate, V, Stroobant, D. Colau, N. Parmentier, et al., Evidence for a tumoral immune resistance mechanism based on tryptophan degradation by indoleamine 2,3-dioxygenase, Nature Medicine 9 (2003) 1269-1274.

1. A method of inducing patient immune tolerance to an antigen comprising:
(a) culturing in vitro
(i) immune cells obtained from blood of the patient,
(ii) an antigen-containing sample, and
(iii) zebularine to form a culture;
(b) enriching dendritic cells in the culture of (a); and
(c) administering the enriched dendritic cells to the patient, thereby inducing immune tolerance in the patient against the antigen.

2. The method of claim 1, wherein the antigen-containing sample comprises a foreign antigen or a patient self antigen.

3. The method of claim 2, wherein the antigen-containing sample comprises a foreign antigen from a transplant donor, thereby inducing immune tolerance in the patient against the foreign antigen.

4. The method of claim 3, wherein inducing immune tolerance in the patient against the foreign antigen comprises inducing immune tolerance in the patient against the transplanted tissue of the donor comprising the foreign antigen.

5. The method of claim 3, wherein inducing immune tolerance in the patient against the foreign antigen comprises inducing immune tolerance in the patient against transplanted beta cells of the donor comprising the foreign antigen.

6. The method of claim 3, wherein the antigen-containing sample comprises a protein extract comprising the foreign antigen from the transplant donor.

7. The method of claim 2, wherein the antigen-containing sample comprises a patient self antigen, thereby inducing immune tolerance in the patient against the self antigen.

8. The method of claim 7, wherein the antigen-containing sample comprises a patient collagen or cartilage sample comprising the self antigen.

9. The method of claim 1, wherein the dendritic cells comprise regulatory plasmacytoid dendritic cells.

10. The method of claim 1, wherein the immune cells comprise white blood cells.

11. The method of claim 1, wherein the patient blood comprises peripheral blood.

12. The method of claim 1, wherein step (a) further comprises culturing the cells with granulocyte-macrophage colony-stimulating factor (GMCSF).

13. The method of claim 1, wherein step (a) further comprises stimulating the cultured immune cells with oligonucleotides.

14. The method of claim 13, further comprising culturing the stimulated cells in the presence of zebularine and histone deactylase (HDAC) inhibitors.

15. The method of claim 1, wherein the patient has an autoimmune disease.

16. The method of claim 15, wherein the autoimmune disease is diabetes type 1.

17. The method of claim 16, wherein he patient is to receive a transplant of beta cells from the donor.

18. The method of claim 15, wherein the autoimmune disease comprises rheumatoid arthritis.

19. A method of inducing IDO in a culture of immune cells obtained from a patient comprising:
(a) isolating immune cells from blood of the patient;
(b) culturing in vitro
(i) the immune cells,
(ii) an antigen-containing sample, and
(iii) zebularine; and
(c) obtaining a cell culture in which IDO is induced.

20. The method of claim 19, wherein the antigen-containing sample comprises a foreign antigen or a patient self antigen.

21. The method of claim 20, wherein the antigen-containing sample comprises a foreign antigen from a transplant donor.

22. The method of claim 21, wherein the antigen-containing sample comprises a protein extract comprising the foreign antigen from the transplant donor.

23. The method of claim 19, wherein the antigen-containing sample comprises a patient self antigen.

24. The method of claim 23, wherein the antigen-containing sample comprises a patient collagen or cartilage sample comprising the self antigen.

25. The method of claim 19, wherein the immune cells comprise white blood cells.

26. The method of claim 19, wherein the patient blood comprises peripheral blood.

27. The method of claim 19, further comprising enriching dendritic cells in the cell culture.

28. The method of claim 27, wherein the dendritic cells comprise regulatory plasmacytoid dendritic cells.

29. The method of claim 19, wherein step (b) further comprises culturing the cells with granulocyte-macrophage colony-stimulating factor (GMCSF).

30. The method of claim 19, wherein step (b) further comprises stimulating the cultured immune cells with oligonucleotides.

31. The method of claim 30, further comprising culturing the stimulated cells in the presence of zebularine and histone deactylase (HDAC) inhibitors.

32. The method of claim 19, wherein the patient has an autoimmune disease.

33. The method of claim 32, wherein the autoimmune disease is diabetes type 1.

34. The method of claim 33, wherein the patient is to receive a transplant of beta cells from the donor.

35. The method of claim 32, wherein the autoimmune disease comprises rheumatoid arthritis.

36. A cell culture comprising
(a) isolated immune cells from blood of a patient who (i) is to receive a transplant or (ii) has an autoimmune disease;
(b) an antigen-containing sample comprising
(i) a foreign antigen from a transplant donor, wherein the patient is to receive a transplant from the donor; or
(ii) a patient self antigen, wherein the patient has an autoimmune disease; and
(c) zebularine;
wherein IDO is induced in said cell culture.

37. The cell culture of claim 36, wherein the antigen-containing sample comprises
(a) a protein extract comprising the foreign antigen from the transplant donor; or
(b) a patient collagen or cartilage sample comprising the self antigen.
